(19) United States
(12) Patent Application Publication
(10) Pub. No.: US 2026/0334842 A1
(43) Pub. Date: Oct. 29, 2026

(54) ELECTRODE PATCH AND ELECTROCARDIOGRAM DETECTION APPARATUS

(71) Applicant: CARDIOCLOUD MEDICAL TECHNOLOGY (BEIJING) CO., LTD., Beijing (CN)

(72) Inventors: Ping WANG, Beijing (CN); Liheng CHEN, Beijing (CN); Jinliang WANG, Beijing (CN)

(73) Assignee: CARDIOCLOUD MEDICAL TECHNOLOGY (BEIJING) CO., LTD., Beijing (CN)

(21) Appl. No.: 19/693,642

(22) Filed: Jun. 1, 2026

Related U.S. Application Data
(63) Continuation of application No. PCT/CN2024/132746, filed on Nov. 18, 2024.

(30) Foreign Application Priority Data
Dec. 1, 2023 (CN) 202311639643.6

(51) Int. Cl. A61B 5/28; A61B 5/259; A61B 5/265; A61B 5/27

(52) U.S. Cl. CPC A61B 5/28; A61B 5/259; A61B 5/265; A61B 5/27

(57) ABSTRACT

The present application provides an electrode patch and an electrocardiogram detection apparatus. The electrode patch includes: an electrically conductive contact unit attached to an object to be detected to collect an electrical signal from the object; an isolation sheet attached to the electrically conductive contact unit to isolate the electrically conductive contact unit from air; a conduction assembly to fix the isolation sheet and the electrically conductive contact unit and connected to a main unit to transmit the electrical signal collected by the electrically conductive contact unit to the main unit; a fabric, provided with an adhesive substance to be attached to the object; an adhesive ring to fix the fabric and adhered to the isolation sheet and the fabric to form a sealed space; and a release paper assembly, adhered to the fabric and the adhesive ring to maintain humidity of the electrically conductive contact unit.

### CROSS-REFERENCE TO RELATED APPLICATIONS

This application is a continuation of International Application No. PCT/CN2024/132746, filed on Nov. 18, 2024, which claims priority to Chinese Patent Application No. 202311639643.6, filed on Dec. 1, 2023. The entire contents of each of the above-referenced applications are incorporated herein by reference.

### TECHNICAL FIELD

The present application relates to the technical field of medical devices, and in particular to an electrode patch and an electrocardiogram detection apparatus.

### BACKGROUND

Electrocardiogram electrodes play a crucial role in various applications such as heart disease examinations, cardiac monitoring, and cardiac surgeries. As an essential part of electrocardiogram detection, they can convert cardiac activities into electrical signals to provide vital diagnostic information and assist in developing treatment plans.

At present, electrocardiogram electrodes on the market are mainly divided into two categories, namely, reusable electrodes and disposable electrodes. The reusable electrodes are generally dry electrodes that require disinfection and moistening before use, for example, clip-on electrodes and suction-cup electrodes for electrocardiogram machines.

The above information disclosed in the background art section is only intended to strengthen the understanding of the background of the present application, and may therefore include information that does not constitute the prior art known to those of those skilled in the art.

### SUMMARY

The present application provides an electrode patch and an electrocardiogram detection apparatus.

According to the first aspect of the present application, at least one embodiment of the present application provides an electrode patch, including: an electrically conductive contact unit, configured to be attached to an object to be detected, so as to collect an electrical signal from the object to be detected; an isolation sheet, configured to be attached to the electrically conductive contact unit, so as to isolate the electrically conductive contact unit from air; a conduction assembly, configured to fix the isolation sheet and the electrically conductive contact unit and configured to be connected to a main unit, so as to transmit the electrical signal collected by the electrically conductive contact unit to the main unit; a fabric, provided with an adhesive substance, so as to be attached to the object to be detected; an adhesive ring, configured to fix the fabric and adhered to the isolation sheet and the fabric, so as to form a sealed space; and a release paper assembly, adhered to the fabric and the adhesive ring, so as to maintain humidity of the electrically conductive contact unit.

For example, in some embodiments of the present application, the electrically conductive contact unit includes: an electrically conductive cloth, configured to collect the electrical signal from the object to be detected, and being a fibrous structure embedded with an electrically conductive material.

For example, in some embodiments of the present application, the electrically conductive material includes at least one of gold, silver, silver chloride, titanium, and graphene.

For example, in some embodiments of the present application, the electrically conductive cloth is provided with a first groove configured to store hydrogel. The hydrogel is configured to provide moisture when the electrically conductive cloth is attached to the object to be detected, so as to moisten the electrically conductive cloth and the object to be detected, thereby reducing contact resistance between the electrically conductive cloth and the object to be detected.

For example, in some embodiments of the present application, the hydrogel includes: water; or water and at least one of vitamin C, hyaluronic acid, sodium pyrrolidonecarboxylate and diluted glycerol.

For example, in some embodiments of the present application, the electrically conductive contact unit further includes: an adhesive mesh, with one side attached to an outer side of the first groove and another side attached to the object to be detected.

For example, in some embodiments of the present application, when the adhesive mesh is attached to the first groove, a proportion of an area of the first groove attached to the object to be detected is greater than a first threshold of a bottom area of the first groove.

For example, in some embodiments of the present application, the isolation sheet includes: an outer plastic layer, attached to the electrically conductive cloth so as to seal the hydrogel; and an inner plastic layer, disposed between the electrically conductive cloth and the adhesive mesh, the inner plastic layer being provided with a first hole to allow the first groove to pass through the first hole and then to be attached to the object to be detected.

For example, in some embodiments of the present application, the conduction assembly includes: a first component and a second component; and wherein the electrically conductive cloth is provided with a second hole, and the outer plastic layer is provided with a third hole, to allow the second component to pass through the second hole and the third hole and then to be connected to the first component, thereby fixing the electrically conductive contact unit and the isolation sheet.

For example, in some embodiments of the present application, the second hole and the third hole are disposed at a central or eccentric position of the electrically conductive contact unit and the isolation sheet.

For example, in some embodiments of the present application, the fabric includes: a first ring and a second ring, wherein: a non-adhesive coating region is between the first ring and the second ring, and an adhesive coating region is disposed on an inner side of the first ring and on an outer side of the second ring; and a sweat wicking ring, disposed between the first ring and the second ring and configured to wick moisture away from a surface of the object to be detected.

For example, in some embodiments of the present application, the fabric is a one-way moisture-wicking fabric.

For example, in some embodiments of the present application, the electrode patch further includes: a sealing gasket, attached to the fabric and the adhesive ring so as to increase airtightness between the fabric and the adhesive ring.

For example, in some embodiments of the present application, the adhesive ring includes: a first adhesive ring, disposed between the inner plastic layer and fabric and attached to the inner plastic layer of the isolation sheet, the fabric, and the sealing gasket; and a second adhesive ring, disposed between the sealing gasket and the release paper assembly and attached to the sealing gasket.

For example, in some embodiments of the present application, the first adhesive ring is provided with a fourth hole; the fabric is provided with a fifth hole; the sealing gasket is provided with a sixth hole; the second adhesive ring is provided with a seventh hole; and the first groove is allowed to pass through the fourth hole, the fifth hole, the sixth hole, and the seventh hole and then to be attached to the object to be detected.

For example, in some embodiments of the present application, an aperture of the fifth hole is greater than apertures of the fourth hole, the sixth hole, and the seventh hole.

For example, in some embodiments of the present application, the release paper assembly includes: release paper, configured to be adhered to the fabric so as to protect an adhesive layer of the fabric; and a sealing cover, configured to be adhered to the adhesive ring so as to protect moisture of the electrically conductive contact unit.

According to a second aspect of the present application, at least one embodiment of the present application provides an electrocardiogram detection apparatus, including the electrode patch according to any description in the first aspect.

For example, in some embodiments of the present application, the electrocardiogram detection apparatus further includes: a main unit, configured to process the electrical signal collected by the electrode patch.

It should be understood that the general description above and the detailed description below are merely for an exemplary purpose, and are not intended to limit the present application.

### BRIEF DESCRIPTION OF THE DRAWINGS

The above and other objects, characteristics, and advantages of the present application will become more obvious by describing the exemplary embodiments in detail with reference to the accompanying drawings. The drawings described below provide only some embodiments of the present application, and are not intended to limit the present application.

FIG. 1 is a schematic diagram of an electrode patch according to an embodiment of the present application;

FIG. 2 is a sectional view of an electrode patch according to an embodiment of the present application;

FIG. 3A is a schematic diagram of a first embodiment of an adhesive mesh of the present application;

FIG. 3B is a schematic diagram of a second embodiment of the adhesive mesh of the present application;

FIG. 3C is a schematic diagram of a third embodiment of the adhesive mesh of the present application;

FIG. 3D is a schematic diagram of a fourth embodiment of the adhesive mesh of the present application;

FIG. 4 is a schematic diagram of a second embodiment of the electrode patch of the present application;

FIG. 5 is a schematic diagram of a fabric according to an embodiment of the present application;

FIG. 6A is a first schematic diagram showing installation of an adhesive ring, a fabric, and a sealing gasket according to an embodiment of the present application;

FIG. 6B is a second schematic diagram showing installation of an adhesive ring, a fabric, and a sealing gasket according to an embodiment of the present application;

FIG. 7A is a first schematic diagram of an electrode patch of the present application;

FIG. 7B is a second schematic diagram of an electrode patch of the present application;

FIG. 7C is a third schematic diagram of an electrode patch of the present application; and

FIG. 8 is a schematic diagram of an electrocardiogram detection apparatus according to an embodiment of the present application.

### REFERENCE SIGNS

1: conduction assembly;
12: first component;
11: second component;
2: isolation sheet;
21: outer plastic layer;
22: inner plastic layer;
3: electrically conductive contact unit;
31: hydrogel;
32: electrically conductive cloth;
33: adhesive mesh;
4: adhesive ring;
5: fabric;
6: sealing gasket;
7: adhesive ring;
8: release paper assembly;
81: release paper; and
82: sealing cover.

### DETAILED DESCRIPTION OF EMBODIMENTS

Exemplary embodiments will be now described in more detail with reference to the accompanying drawings. However, these exemplary embodiments can be implemented in a variety of forms and should not be understood as being limited to the embodiments described herein. On the contrary, these embodiments are provided such that the present application will become comprehensive and complete, and the conception of the exemplary embodiments is fully communicated to those skilled in the art. Identical reference signs in the drawings represent identical or similar parts, and their repeated description will be omitted accordingly.

The described features, structures, or characteristics can be combined in one or more embodiments in any appropriate manner. In the description below, many specific details are provided to thus provide full understanding of the embodiments of the present disclosure. However, those skilled in the art will appreciate that the technical solutions of the present application can be practiced without one or more of these specific details, or other methods, constituent elements, materials, apparatuses or the like can be used. In these cases, the well-known structures, methods, apparatuses, implementations, materials or operations will not be shown or described in detail.

The flowchart shown in the accompanying drawings is only for an illustrative purpose, and does not have to include all the content and operations or steps, nor does it have to be executed in the described order. For example, some operations or steps may be broken down, while other operations or steps may be combined or partially combined. Therefore, the actual execution order may vary according to actual conditions.

The terms "first", "second" and the like in the description and claims as well as in the accompanying drawings of the present application are used to distinguish different objects, and are not intended to describe a specific order. Furthermore, the terms “include” and “have” and any variations thereof are intended to cover non-exclusive inclusions. For example, a process, method, system, product or device including a series of steps or units is not limited to the listed steps or units, but optionally further includes unlisted steps or units, or optionally further includes other steps or units inherent to the stated process, method, product or device.

Those skilled in the art may understand that the accompanying drawings are only schematic diagrams of exemplary embodiments, and the modules or flow processes in the accompanying drawings are not necessarily required for implementing the present application, and thus cannot be used to limit the protection scope of the present application.

Although the reusable electrodes are lower in cost and easier to operate, they are only suitable for short-term fragmented collection of electrocardiogram signals, and easily lead to cross-contamination in case of insufficient disinfection. The disposable electrodes are mainly used for long-term electrocardiogram monitoring, with firm adherence and good electrical conductivity, but easily lead to skin allergies. And, in case of prolonged use of the disposable electrocardiogram electrodes, the adhesive on the electrode patches and the high-concentration electrolytes in the electrically conductive gel are prone to causing skin allergies and contact dermatitis. Furthermore, the risk of allergic reactions caused by the disposable electrocardiogram electrodes is directly proportional to the duration of adherence, and the longer the adherence, the higher the occurrence of the risk. In addition, the electrically conductive gel on the disposable electrodes is prone to peeling off when exposed to heavy sweating, leading to poor contact and thus affecting the collection of electrocardiogram signals.

Although the electrocardiogram electrodes are important in the diagnosis and treatment of heart disease, the frequent replacement of the electrocardiogram electrodes may lead to waste of electrode patches and increase in costs.

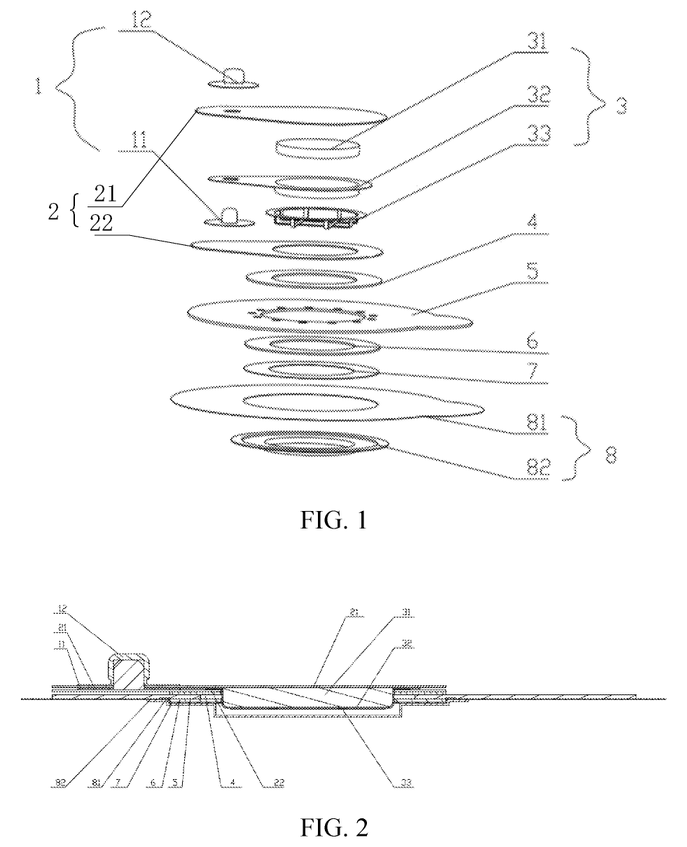

As shown in FIGS. 1 and 2, the electrode patch includes a conduction assembly 1, an isolation sheet 2, an electrically conductive contact unit 3, an adhesive ring 4, an adhesive ring 7, a fabric 5, and a release paper assembly 8.

The conduction assembly 1 is configured to fix the isolation sheet 2 and the electrically conductive contact unit 3. The conduction assembly 1 is further configured to be connected to a main unit (not shown in the drawings) so as to transmit an electrical signal collected by the electrically conductive contact unit 3 to the main unit.

The electrically conductive contact unit 3 is configured to be attached to an object to be detected to collect the electrical signal. For example, the electrically conductive contact unit is attached to a human body to collect electrocardiogram signals from the human body.

The electrically conductive contact unit 3 includes an electrically conductive cloth 32 and an adhesive mesh 33. The electrically conductive cloth is configured to collect the electrical signal from the object to be detected.

According to some embodiments, the electrically conductive cloth is a fibrous structure embedded with an electrically conductive material, and the electrically conductive material includes at least one of gold, silver, silver chloride, titanium, and graphene. The electrically conductive material can be plated on the surface of the fibrous structure. For example, silver is plated onto the surface of the electrically conductive cloth to enable the electrically conductive cloth to collect and transmit the electrical signal.

For another example, the electrically conductive cloth is a 30-50D warp-knitted or weft-knitted fabric with a weight of 90-120g/m², and is made of silver with the antibacterial rate of greater than 99 % and the surface resistance of less than 1 Ω/cm. In this case, the electrically conductive cloth has excellent electrical conductivity. Moreover, the electrically conductive cloth is subjected to anti-oxidation treatment, making it resistant to sweat corrosion.

The electrically conductive cloth 32 is provided with a first groove, and the first groove is attached to the object to be detected. The first groove is configured to store hydrogel 31, so as to ensure that the moisture from the hydrogel 31 permeates the electrically conductive cloth 32 when the electrode patch is in use, thereby keeping the electrically conductive cloth 32 and the object to be detected moist. Therefore, when the electrode patch is in use, the contact resistance between the electrically conductive cloth and the object to be detected can be reduced to enable the electrode patch to more effectively collect the electrical signal, thereby ensuring the accuracy of electrical signal collection by the electrically conductive contact unit 3.

According to some embodiments, the hydrogel contains pure water or water, and other skin protective ingredients, such as vitamin C, hyaluronic acid, sodium pyrrolidone carboxylate and/or diluted glycerol. Wherein, the water content of the hydrogel is 50 %-90 %. This is provided only as an example in the present application, and the specific water content and composition of the hydrogel are not limited thereto. The hydrogel provided in the present application is substantially free of electrolyte ingredients so as to reduce the risk of skin allergies in the object to be detected.

The adhesive mesh 33 is in the form of double-sided adhesive tape, with one side attached to the outer side of the first groove and another side attached to the object to be detected. In this way, when the electrode patch is adhered to the object to be detected, the electrode patch is fixed to the object to be detected by adhering the adhesive mesh 33 to the surface of the object to be detected, thereby maintaining the stability of the electrode patch.

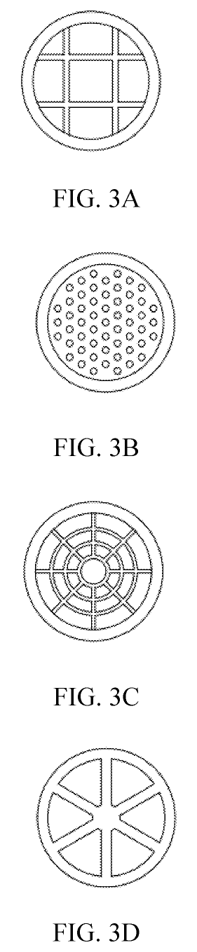

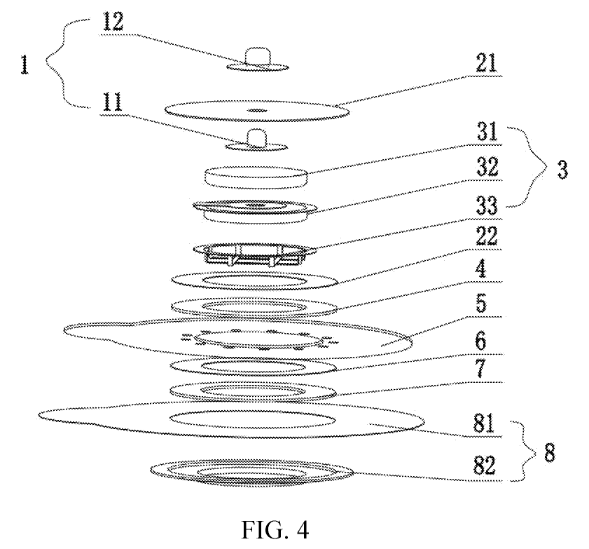

According to some embodiments, the adhesive mesh 33 may take various shapes. FIGS. 3A - 3D illustrate the shapes of the adhesive mesh in exemplary embodiments. For example, the adhesive mesh is in the form of a strip as shown in FIG. 3A, a dot as shown in FIG. 3B, a mesh as shown in FIG. 3C, a ring as shown in FIG. 3D, or a combination of the aforementioned forms.

According to some embodiments, when the adhesive mesh 33 is attached to the first groove, the first groove is partially exposed. That is, the area of the first groove attached to the object to be detected is greater than a first threshold of a bottom area of the first groove, so as to ensure the effectiveness of electrical signals collected by conductive cloth 32. Wherein, the first threshold may be set arbitrarily. For example, the first threshold is 50 %, meaning that the area of the part of the first groove attached to the object to be detected needs to be greater than 50 % of the bottom area of the first groove.

The isolation sheet 2 is configured to be adhered to the electrically conductive contact unit 3 so as to isolate the electrically conductive contact unit from air.

The isolation sheet 2 includes an outer plastic layer 21 and an inner plastic layer 22. Wherein, the outer plastic layer is attached to the electrically conductive cloth. That is, the outer plastic layer 21 is disposed on top of the electrically conductive cloth 32 to seal the electrically conductive cloth 32 and the hydrogel 31 stored within the electrically conductive cloth 32, and maintain moisture retention of the conductive cloth 32 for a longer time. The inner plastic layer is disposed between the electrically conductive cloth and the adhesive mesh. That is, the inner plastic layer is disposed beneath the electrically conductive contact unit 3 when the electrically conductive contact unit 3 is attached to the object to be detected. Moreover, the inner plastic layer 22 is provided with a first hole, enabling the first groove of the electrically conductive cloth 32 to pass through the first hole and then to be attached to the object to be detected.

As shown in FIG. 2, the outer plastic layer 21 is attached to the electrically conductive cloth 32, the first groove of the electrically conductive cloth 32 is filled with hydrogel 31, and the adhesive mesh 33 is attached beneath the first groove of the electrically conductive cloth 32.

According to some embodiments, the outer plastic layer 21 and the inner plastic layer 22 may be made of polyvinyl chloride (PVC) or other biocompatible plastics. These are provided only as an example in the present application, and the present application is not limited thereto.

According to some embodiments, the outer plastic layer 21 and the inner plastic layer 22 may be welded using ultrasonic welding to seal and fix the electrically conductive contact unit 3.

The conduction assembly 1 includes a first component 12 and a second component 11. Wherein the electrically conductive cloth 32 is provided with a second hole, and the outer plastic layer 21 is provided with a third hole, to allow the second component 11 to pass through the second hole and the third hole and then to be connected to the first component, thereby fixing the electrically conductive contact unit 3 and the isolation sheet 2 and transmitting the electrical signal collected by the electrically conductive contact unit 3 to the main unit.

According to some embodiments, the second hole in the electrically conductive cloth 32 and the third hole in the outer plastic layer 21 may be positioned eccentrically. That is, the conduction assembly 1 is positioned eccentrically, as shown in FIG. 1. The second hole in the electrically conductive cloth 32 and the third hole in the outer plastic layer 21 may be positioned centrally. That is, the conduction assembly 1 is positioned centrally, as shown in FIG. 4.

The fabric 5 is provided with an adhesive substance so as to be attached to the object to be detected. The fabric is provided with a fifth hole to allow the first groove of the electrically conductive cloth 32 to be attached to the object to be detected via the fifth hole.

According to some embodiments, the surface of the fabric 5 attached to the object to be detected is adhesive-coated.

According to some embodiments, when conventional electrode patches are attached to an object to be detected, excessive sweating on the subject will trap sweat inside the patch due to full adhesive coating on the skin-contacting side, leading to discomfort to the object.

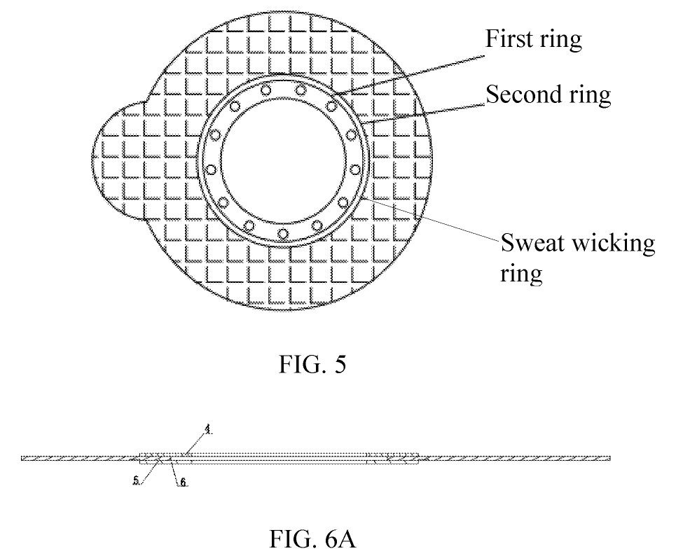

Therefore, the present application proposes providing a first ring and a second ring on the fabric 5, as shown in FIG. 5. Wherein, an adhesive coating region is disposed on an inner side of the first ring and on an outer side of the second ring, i.e., coating an adhesive on the inner side of the first ring and on the outer side of the second ring; and a non-adhesive coating region is between the first ring and the second ring, i.e., no adhesive coated between the first ring and the second ring. In this way, when the electrode patch is attached to the object to be detected, the moisture is wicked away from the surface of the object to be detected. For example, the sweat is wicked from a surface attached to the object to be detected, to a surface away from the object to be detected.

According to some embodiments, the fabric 5 is further provided with a sweat wicking ring, as shown in FIG. 5. The sweat wicking ring is disposed between the first ring and the second ring and is configured to wick the sweat from a surface attached to the object to be detected, to a surface away from the object to be detected, thereby reducing the risk of allergies when the electrode patch is applied to the object to be detected.

According to some embodiments, the fabric 5 is a one-way moisture-wicking fabric. In this way, when the electrode patch is attached to the object to be detected, the sweat can be wicked from a surface attached to the object to be detected, to a surface away from the object to be detected, thereby improving wearing comfort of the electrode patch.

According to some embodiments, a material for the fabric 5 includes cotton, linen, silk, and blended synthetic fibers. When the electrode patch is intended for short-term use, conventional materials such as non-woven fabric or foam may also be used to reduce the production cost of the electrode patch.

According to some embodiments, the electrode patch may further include a sealing gasket 6. The sealing gasket 6 is attached to the fabric 5, the adhesive ring 4, and the adhesive ring 7, to ensure the airtightness between the sealing gasket 6 and the adhesive rings 4 and 7.

According to some embodiments, the sealing gasket 6 is made of biocompatible plastic. The sealing gasket 6 is provided with a sixth hole, allowing firs groove of the electrically conductive cloth 32 to be attached to the object to be detected via the sixth hole.

The adhesive rings of the present application include a first adhesive ring 4 and a second adhesive ring 7, which are configured to fix the fabric 5 and the sealing gasket 6. Wherein, the first adhesive ring 4 is made of a double-sided adhesive material, and is disposed between the inner plastic layer 22 and the fabric 5 to connect the inner plastic layer 22, the fabric 5, and the sealing gasket 6. The second adhesive ring 7 is made of a double-sided adhesive material and is disposed between the sealing gasket 6 and the release paper assembly 8 and attached to the sealing gasket 6. The first adhesive ring 4 is provided with a fourth hole, and the second adhesive ring 7 is provided with a seventh hole, such that the first groove of the electrically conductive cloth 32 passes through the fourth hole and the seventh hole, and then is attached to the object to be detected.

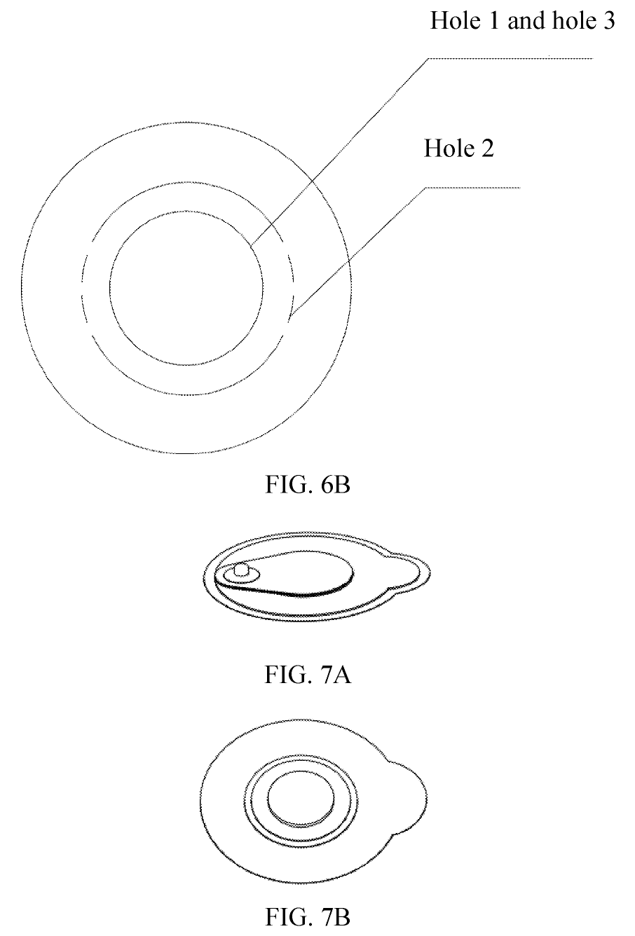

According to some embodiments, as shown in FIGS. 6A and 6B, the aperture of the fifth hole of the fabric 5 is greater than the aperture of the fourth hole of the first adhesive ring 4, the seventh hole of the second adhesive ring 7, and the sixth hole of the sealing gasket 6, and the latter three apertures are equal in size. Wherein, the hole 2 is the fifth hole of the fabric 5, the hole 1 is the fourth hole of the first adhesive ring 4 or the seventh hole of the second adhesive ring 7, and the hole 3 is the sixth hole of the sealing gasket 6. Wherein, the outer diameters of the adhesive ring 4, the sealing gasket 6, and the adhesive ring 7 are the same, and are identical to the size of the first ring in the fabric 5. Wherein, the fabric 5 is provided with a small hole between the first ring and the fifth hole. In this way, part of the adhesive ring 4 passes through the fifth hole of the fabric 5 and is adhered to the sealing gasket 6, while another part of the adhesive ring 4 passes through the small hole in the fabric 5 and is adhered to the sealing gasket 6, thereby ensuring the reliability and airtightness of the adherence among the adhesive ring 4, the fabric 5, and the sealing gasket 6. As shown in FIG. 5, the small hole of the fabric 5 refers to the small hole on the inner side of the first ring.

Hence, the inner plastic layer 22, the first adhesive ring 4, the fabric 5, the sealing gasket 6, and the second adhesive ring 7 are combined and adhered to form a sealed space.

The release paper assembly 8 is adhered to the electrically conductive contact unit 3, the fabric 5, and the second adhesive ring 7 to form a sealed space, so as keep the electrically conductive contact unit moist. Wherein, the release paper assembly 8 includes release paper 81 and a sealing cover 82. The release paper 81 is adhered to the fabric 5 so as to protect an adhesive layer of the fabric 5. Moreover, when the electrode patch is used, the release paper 81 can be removed conveniently. The sealing cover 82 is configured to cover the electrically conductive contact unit 3 and is adhered to the second adhesive ring 7, such that a sealed space is formed for the electrode patch to prevent moisture in the hydrogel 31 from evaporation, thereby protecting the humidity of the electrically conductive contact unit.

According to some embodiments, the sealing cover 82 and the release paper 81 are welded together. The sealing cover 82 is made of plastic PE or PET.

The electrode patch provided by the present application is simple in structure, in which the hydrogel is added to the electrically conductive contact unit to keep it moist, and the skin resistance can be reduced when the electrode patch is in use, thereby enabling more effective collection of electrical signals; the part of the electrically conductive contact unit in contact with the object to be detected is fixed using the adhesive mesh, thereby enhancing the positional stability of the electrode patch; and the one-way moisture-wicking fabric is used to enable rapid sweat wicking, thereby increasing the wearer comfort during the use of the electrode patch and reducing the risk of allergies.

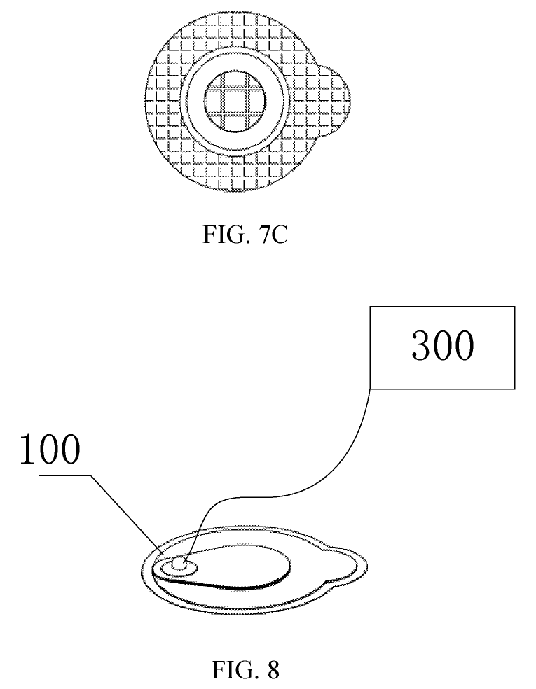

As shown in FIG. 7A, it is a front view of the electrode patch, i.e., the side facing away from the object to be detected. As shown in FIG. 7B, it is a rear view of the electrode patch, i.e., the side attached to the object to be detected. As shown in FIG. 7C, it is a schematic diagram of the side of the electrode patch attached to the object to be detected after the release paper assembly is removed.

The present application further provides an electrocardiogram detection apparatus configured to collect electrocardiogram signals from an object to be detected. As shown in FIG. 8, the electrocardiogram detection apparatus includes the electrode patch 100 as described above and a main unit 300. The electrode patch is connected to the main unit, and the main unit 300 is configured to process electric signals collected by the electrode patch 100.

The electrode patch provided by the present application is simple in structure, in which the hydrogel is added to the electrically conductive contact unit to keep the electrically conductive contact unit moist, such that the skin resistance can be reduced when the electrode patch is in use, thereby enabling more effective collection of electrical signals; the part of the electrically conductive contact unit in contact with the object to be detected is fixed using the adhesive mesh, thereby enhancing the positional stability of the electrode patch; and the one-way moisture-wicking fabric is used to enable rapid sweat wicking, thereby increasing the wearer comfort during the use of the electrode patch and reducing the risk of allergies.

It should be clearly understood that the present application describes how to develop and use specific examples, but the present application is not limited to any details of these examples. On the contrary, the principles of these examples can be applied to many other embodiments based on the guidance in the disclosure of the present application.

Furthermore, it should be noted that the above drawings are merely schematic illustration of the processes included in the method according to the exemplary embodiments of the present application, and are not for limiting purposes. It is readily understood that the processes shown in the drawings above do not indicate or limit the time sequence of these processes. In addition, it is also readily understood that these processes can be performed synchronously or asynchronously, for example, in a plurality of modules.

The above provides detailed presentation and description of the exemplary embodiments of the present application. It should be understood that the present application is not limited to the detailed structure, arrangement mode, or implementation method described here. On the contrary, the present application is intended to encompass various modifications and equipment configurations included within the spirit and scope of the attached claims.

What is claimed is:

1. An electrode patch, comprising:
an electrically conductive contact unit, configured to be attached to an object to be detected, so as to collect an electrical signal from the object to be detected;
an isolation sheet, configured to be attached to the electrically conductive contact unit, so as to isolate the electrically conductive contact unit from air;
a conduction assembly, configured to fix the isolation sheet and the electrically conductive contact unit and configured to be connected to a main unit, so as to transmit the electrical signal collected by the electrically conductive contact unit to the main unit;
a fabric, provided with an adhesive substance, so as to be attached to the object to be detected;
an adhesive ring, configured to fix the fabric and adhered to the isolation sheet and the fabric, so as to form a sealed space; and
a release paper assembly, adhered to the fabric and the adhesive ring, so as to maintain humidity of the electrically conductive contact unit.

2. The electrode patch according to claim 1, wherein the electrically conductive contact unit comprises:
an electrically conductive cloth, configured to collect the electrical signal from the object to be detected, and being a fibrous structure embedded with an electrically conductive material.

3. The electrode patch according to claim 2, wherein the electrically conductive material comprises at least one of gold, silver, silver chloride, titanium, and graphene.

4. The electrode patch according to claim 2, wherein the electrically conductive cloth is provided with a first groove configured to store hydrogel, the hydrogel is configured to provide moisture when the electrically conductive cloth is attached to the object to be detected, so as to moisten the electrically conductive cloth and the object to be detected, thereby reducing contact resistance between the electrically conductive cloth and the object to be detected.

5. The electrode patch according to claim 4, wherein the hydrogel comprises:
water; or
water and at least one of vitamin C, hyaluronic acid, sodium pyrrolidonecarboxylate, and diluted glycerol.

6. The electrode patch according to claim 4, wherein the electrically conductive contact unit further comprises:
an adhesive mesh, with one side attached to an outer side of the first groove and another side attached to the object to be detected.

7. The electrode patch according to claim 4, wherein when the adhesive mesh is attached to the first groove, a proportion of an area of the first groove attached to the object to be detected is greater than a first threshold of a bottom area of the first groove.

8. The electrode patch according to claim 6, wherein the isolation sheet comprises:
an outer plastic layer, attached to the electrically conductive cloth so as to seal the hydrogel; and
an inner plastic layer, disposed between the electrically conductive cloth and the adhesive mesh, the inner plastic layer being provided with a first hole to allow the first groove to pass through the first hole and then to be attached to the object to be detected.

9. The electrode patch according to claim 8, wherein the conduction assembly comprises:
a first component and a second component;
wherein the electrically conductive cloth is provided with a second hole, and the outer plastic layer is provided with a third hole, to allow the second component to pass through the second hole and the third hole and then to be connected to the first component, thereby fixing the electrically conductive contact unit and the isolation sheet.

10. The electrode patch according to claim 9, wherein the second hole and the third hole are disposed at a central or eccentric position of the electrically conductive contact unit and the isolation sheet.

11. The electrode patch according to claim 1, wherein the fabric comprises:
a first ring and a second ring, wherein:
a non-adhesive coating region is between the first ring and the second ring, and
an adhesive coating region is disposed on an inner side of the first ring and on an outer side of the second ring; and
a sweat wicking ring, disposed between the first ring and the second ring and configured to wick moisture away from a surface of the object to be detected.

12. The electrode patch according to claim 11, wherein the fabric is a one-way moisture-wicking fabric.

13. The electrode patch according to claim 1, further comprising:
a sealing gasket, attached to the fabric and the adhesive ring so as to increase airtightness between the fabric and the adhesive ring.

14. The electrode patch according to claim 13, wherein the adhesive ring comprises:
a first adhesive ring, disposed between the inner plastic layer and the fabric and attached to the inner plastic layer of the isolation sheet, the fabric, and the sealing gasket; and
a second adhesive ring, disposed between the sealing gasket and the release paper assembly and attached to the sealing gasket.

15. The electrode patch according to claim 14, wherein
the first adhesive ring is provided with a fourth hole;
the fabric is provided with a fifth hole;
the sealing gasket is provided with a sixth hole;
the second adhesive ring is provided with a seventh hole; and
the first groove is allowed to pass through the fourth hole, the fifth hole, the sixth hole, and the seventh hole and then to be attached to the object to be detected.

16. The electrode patch according to claim 15, wherein
an aperture of the fifth hole is greater than apertures of the fourth hole, the sixth hole, and the seventh hole.

17. The electrode patch according to claim 1, wherein the release paper assembly comprises:
release paper, configured to be adhered to the fabric so as to protect an adhesive layer of the fabric; and
a sealing cover, configured to be adhered to the adhesive ring so as to protect moisture of the electrically conductive contact unit.

18. An electrocardiogram detection apparatus, comprising the electrode patch according to claim 1.

19. The electrocardiogram detection apparatus according to claim 18, further comprising:
a main unit, configured to process the electrical signal collected by the electrode patch.
